(19) United States
(12) Patent Application Publication
(10) Pub. No.: US 2024/0315673 A1
(43) Pub. Date: Sep. 26, 2024

(54) BREATHING PATTERN EXTRACTION

(71) Applicant: KONINKLIJKE PHILIPS N.V., EINDHOVEN (NL)

(72) Inventors: Shyam Bharat, Arlington, MA (US); Sven Peter Prevrhal, Hamburg (DE); Jochen Kruecker, Andover, MA (US); Seyedali Sadeghi, Melrose, MA (US); Claudia Errico, Medford, MA (US)

(21) Appl. No.: 18/574,975

(22) PCT Filed: Jun. 29, 2022
(86) PCT No.: PCT/EP2022/067935

§ 371 Date: Dec. 28, 2023

Related U.S. Application Data
(60) Provisional application No. 63/216,694, filed on Jun. 30, 2021.

(51) Int. Cl. A61B 8/08; A61B 5/08; A61B 8/00; G06T 7/00; G16H 15/00

(52) U.S. Cl. CPC A61B 8/5223; A61B 5/0816; A61B 8/543; G06T 7/0012; G16H 15/00; G06T 2207/10132; G06T 2207/20084

(57) ABSTRACT

A system, method, and computer-readable medium are disclosed for extracting breathing pattern data from ultrasound images for aiding downstream clinical patient management, including detecting a trigger event indicative of a breathing pattern from at least one of an audio-based trigger and an image-based trigger from an ultrasound video stream. The presently disclosed technique may further include identifying a breathing pattern in the ultrasound video stream responsive to detection of the trigger event. The presently disclosed technique may also include extraction of at least one breathing-related parameter and the generation of a record with the at least one breathing-related parameter.

### FIELD OF THE INVENTION

The presently disclosed techniques relate to the field of clinical patient management, and more particularly to automated extraction of breathing patterns from ultrasound for aiding downstream clinical patient management.

### BACKGROUND

The ability to efficiently perform imaging exams such as ultrasound, MRI etc. is often dependent on the ability of the patient to comply with instructions of the staff or technologists conducting the exam. The inability of the patient to perform tasks such as breath-holds for a certain duration, taking deep breaths, keeping still, positioning the body in a certain manner etc. can not only prolong the duration of the exam, but can also limit its diagnostic value in some scenarios. Pre-existing conditions in a patient can also impact their ability to comply with exam instructions. For example, if a patient with Parkinson's disease comes in for a peripheral vascular scan, there is a certain level of difficulty associated with the scan due to the repetitive motion of the legs. A patient with fibromyalgia may not be able to assume certain scan positions for US exams.

### SUMMARY

The presently disclosed techniques leverage an initial upstream ultrasound imaging exam to automatically extract parameters (information related to the breathing patterns) of the patient. The extracted parameters are used to auto-populate a report that is editable by the imaging tech, before being pushed to a central database/server that is accessible by subsequent downstream imaging (US/MR/other) or other procedures on that patient.

The initial ultrasound exam on a patient can reveal many attributes related to the patient's ability to comply with exam requirements, such as breathing patterns. The imaging staff typically verbally communicate instructions to the patient (e.g., hold your breath), which can be used as triggers to initiate ultrasound video processing algorithms that identify the start and stop of the breath-hold based on the lack of motion of anatomical structures in the real-time video feed. Inertial measurement unit (IMU) or electromagnetic (EM) sensors on the ultrasound probe are used to filter out probe motion in the algorithm. The presently disclosed techniques may also include an artificial intelligence (AI) model(s) to estimate breathing-related metrics such as breath-hold duration, breathing type, rate, and impact on organ motion.

According to a first aspect of the presently disclosed techniques a system is provided for extracting breathing pattern data from ultrasound images for aiding downstream clinical patient management. The system comprises a detection device configured to detect a trigger event indicative of a specific point of a specific breathing pattern. The detection device may be a microphone and the triggering even may be a verbal direction, such as “take a deep breath and hold it”. An ultrasound video stream such as an ultrasound image stream can be obtained. The ultrasound image stream may be obtained in real-time from an ultrasound probe, or the ultrasound video stream may be reviewed after an examination. A image analysis module initiates a video processing algorithm that looks for corresponding features, such as features corresponding to a specific point in a breathing pattern in the ultrasound image stream responsive to detection of the trigger event, for example to identify movement consistent with a rapid inhalation. The image analysis module then extracts at least one breathing-related parameter from the image stream. A populate algorithm auto-populates a report with the extracted parameter(s), wherein the report is editable by an imaging technologist.

According to one embodiment, the video processing algorithm comprising an artificial intelligence (AI) algorithm trained to estimate different breathing-related parameters specific to the scan or probe maneuver in question.

According to one embodiment, the artificial intelligence algorithm is a model-based regression algorithm. According to one embodiment, the detection device is a microphone and the triggering event is a word or works spoken by the imaging technologist instructing a patient to perform a specific breathing pattern.

According to one embodiment, the detection device is the image analysis module, and the trigger event is a specific motion in the video stream. According to one embodiment, the image analysis module receives tracking data for an ultrasound probe synchronized with the video stream and the image analysis module filters out probe motion from the video stream.

According to one embodiment, the tracking data is generated by an inertial measuring unit (IMU) integral with the ultrasound probe. Alternatively, the tracking data may be generated by electromagnetic tracking coils integral with the ultrasound probe.

According to one embodiment, the estimated breathing parameters comprise breath hold duration. According to one embodiment, the system further comprises an artificial intelligence algorithm trained to estimate heartbeat-related parameters.

According to a second aspect of the presently disclosed techniques, a method is provided for extracting breathing pattern data from ultrasound images for aiding downstream clinical patient management. The method comprises the steps of, detecting a trigger event indicative of a specific point of a specific breathing pattern, obtaining an ultrasound video stream, initiating a video processing algorithm that looks for corresponding features in the ultrasound video stream responsive to detection of the trigger event, estimating at least one breathing-related parameter using a trained artificial intelligence algorithm, and auto-populating a report with the at least one breathing-related parameter.

According to one embodiment, the method further comprises the step of uploading the report to an electronic medical record (EMR) system. According to a third aspect of the presently disclosed techniques, a computer program product is provided. The computer program product comprises a machine-readable media having encoded thereon program code executable by a processor to perform the steps of, detecting a trigger event indicative of a specific point of a specific breathing pattern, obtaining an ultrasound video stream, initiating a video processing algorithm that looks for at least one feature corresponding to the specific point of the breathing pattern in the ultrasound video stream responsive to detection of the trigger event, estimating at least one breathing-related parameter using a trained artificial intelligence algorithm, and auto-populating a report with the at least one breathing-related parameter.

According to one embodiment, the program code is executable by the processor to perform the further step of uploading the report to an electronic medical record (EMR) system. The term “processor”, when used herein shall mean a single processor or a plurality of processors that may be interconnected through hardwiring or wireless connection or may be in communication through a network. The processors may be single core or multi-core processors.

The term “memory”, when used herein, shall mean a machine-readable medium that is either integral with the processor, such as in a workstation or general-purpose computer, or external to processor, such as an external hard drive, cloud storage, or a removable memory device. The medium can be an electronic, magnetic, optical, electromagnetic, infrared, or semiconductor system (or apparatus or device). Examples of a computer-readable medium include a semiconductor or solid-state memory, magnetic tape, a removable computer diskette, a random access memory (RAM), a read-only memory (ROM), a rigid magnetic disk, and an optical disk. Current examples of optical disks include compact disk-read only memory (CD-ROM), compact disk-read/write (CD-R/W) and DVD.

The term “display”, when used herein, shall mean a human viewable computer interface for presenting image data or streams with or without additional images or data as stationary or moving pictures connected to the processor via video graphics array (VGA), digital visual interface (DVI), high-definition multimedia interface (HDMI), low-voltage differential signaling (LVDS) or other proprietary connectors and signals. Examples of currently used displays include liquid crystal displays, light emitting diode displays, plasma displays.

The term “and/or”, when used herein, shall mean only the first possibility, only the second possibility, only the third possibility, and so forth as well as any combination of the listed possibilities. For example, the phrase A, B, and/or C can be any of: only A, only B, only C, A and B, A and C, B and C, or A, B, and C.

The term “image interpretation”, when used herein, shall mean visual review, image manipulation, spatial measurement, temporal measurement, and/or the use of any other imaging tool for identifying characteristics from image data for the purpose of determining medically relevant conditions or making diagnoses.

The term “artificial intelligence algorithm”, when used herein shall mean computer code that can take data (typically in real-time) from multiple sources and take actions (such as making predictions) based on the data and principles, such as minimizing error, based on self-learning.

The term “parameter”, when used herein shall mean a numerical or other measurable factor forming one of a set that defines a system or sets the conditions of its operation. A breathing-related parameter means a measurable factor indicative of a specific breathing pattern for a specific patient.

### BRIEF DESCRIPTION OF THE DRAWINGS

The features and advantages of the presently disclosed techniques will be more clearly understood from the following detailed description of the preferred embodiments when read in connection with the accompanying drawing. Included in the drawing are the following figures:

FIG. 1 is a block diagram of a system for automated extraction of breathing patterns from ultrasound for aiding downstream clinical patient management according to an exemplary embodiment of the presently disclosed techniques.

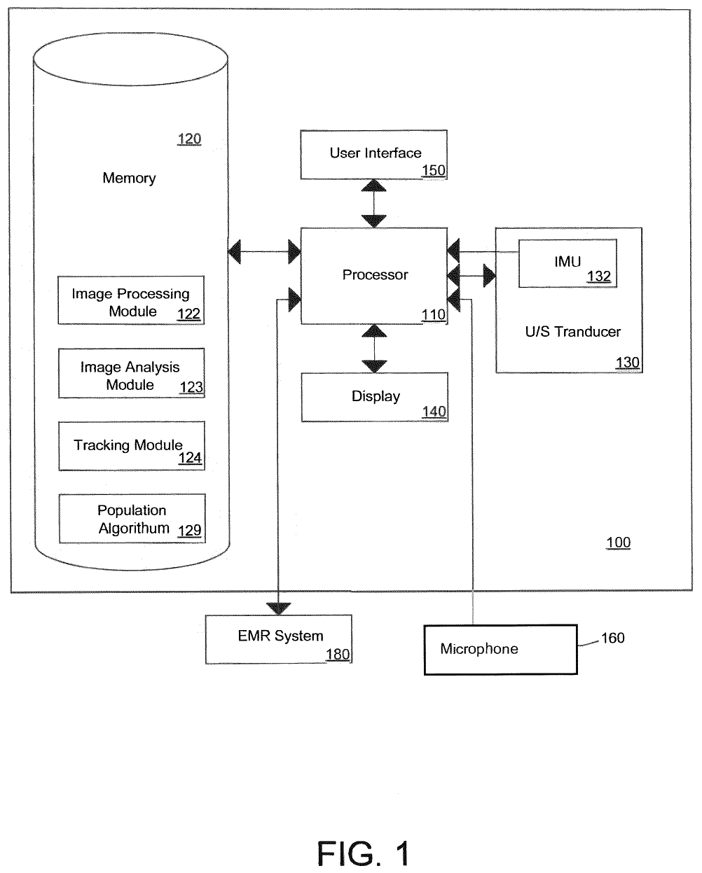

FIG. 2 is a flow diagram of a method for automated extraction of breathing patterns from ultrasound for aiding downstream clinical patient management according to an exemplary embodiment of the presently disclosed techniques.

FIG. 3 is a block diagram of an artificial intelligence algorithm for estimating breathing-related parameters from an ultrasound image stream according to an exemplary embodiment of the presently disclosed techniques.

FIG. 4 is a block diagram of an artificial intelligence algorithm for estimating heartbeat-related parameters from an ultrasound image stream according to an exemplary embodiment of the presently disclosed techniques.

FIG. 5 is a block diagram showing a method for using IMU tracking data to filter intentional ultrasound transducer movement from an ultrasound image stream according to an exemplary embodiment of the presently disclosed techniques.

### DETAILED DESCRIPTION

The presently disclosed techniques provides a method, system and program product for extracting breathing pattern data from ultrasound images for aiding downstream clinical patient management.

According to one embodiment of the presently disclosed techniques, an ultrasound system 100 is configured to extract breathing pattern data (parameters) and provide the breathing data for downstream clinical patient management. The ultrasound system comprises an ultrasound transducer 130 configured to provide an ultrasound pulse, receive an ultrasound echo, and output ultrasound data corresponding to the ultrasound echo. The ultrasound transducer is operably connected to a processor 110 configured to process the ultrasound data to provide imaging data, which is presented on a display 140.

It should be understood, that while the exemplary embodiments described herein use a live image stream and the system includes the ultrasound transducer, the presently disclosed techniques can also be practiced using a stored image stream, wherein the system would not include the ultrasound transducer.

The processor 110 is also operably connected to a memory 120. The memory may comprise one memory media or may comprise a plurality of different memory media that may be interconnected. The memory has encoded thereon, a plurality of software programs of instruction or program codes that are executable by the processor to perform functions which will be described hereafter.

According to one embodiment, an image processing module 122 is stored on memory 120. The image processing module receives imaging data from the ultrasound transducer 130 and generates an ultrasound scan 222. The ultrasound scan 222 is an image or image stream that is presented on the display 140.

According to one embodiment, an image analysis module 123 is stored on memory 120. The image analysis module 123 analyzes the ultrasound scan 222 or image stream and determines imaging parameters related to breathing parameters, such as rapid movement and landmarks entering or leaving the ultrasound scan. The image analysis module 123 extracts feature motion data 223 indicative of motion of features in the ultrasound images.

In one embodiment, a tracking module 124 is stored on memory 120, which calculates the position and orientation of the ultrasound transducer 130 based on tracking data 224. The tracking data 224 can be provided by an inertial measurement unit (IMU) 132 integral to the ultrasound transducer 130, or an electromagnetic (EM) tracking system, or any other positional tracking system. The position, orientation, and motion of the ultrasound transducer 130 can be calculated from the tracking data.

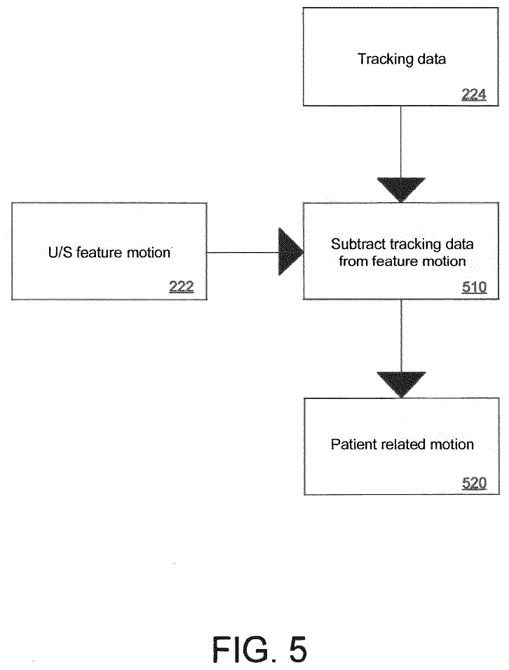

As shown in FIG. 5, the tracking module 124 obtains tracking data 224 from the IMU 132 and ultrasound feature motion from the image analysis module 123. The tracking module subtracts the tracking data from the ultrasound feature motion (step 510) and provides the patient related motion (step 520).

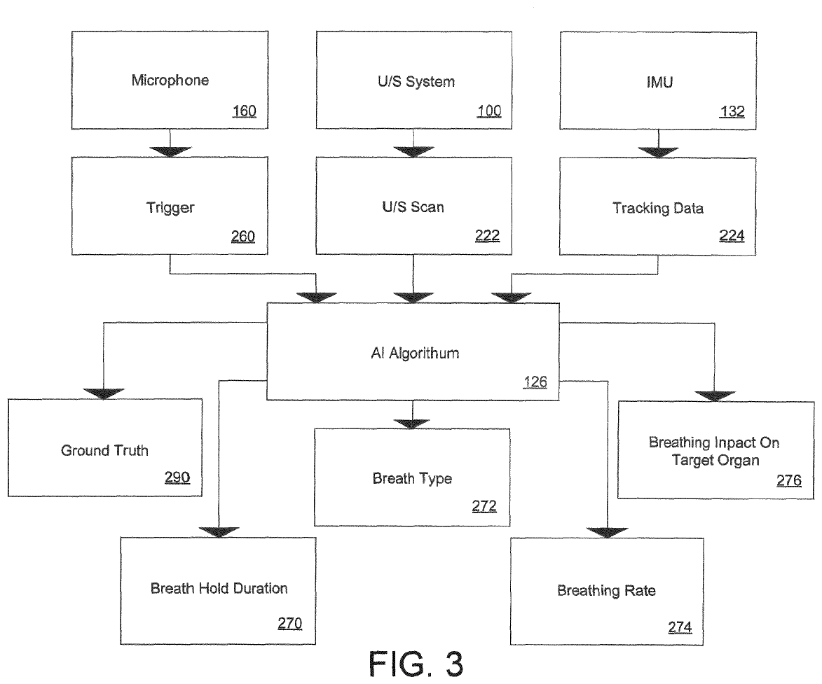

According to one embodiment, the image analysis module 123 comprises an artificial intelligence (AI) algorithm, breathing 126 for estimating breathing related parameters, as shown in FIG. 3. The AI algorithm, breathing 126 is a regression-based reinforcement learning (RL) algorithm. That means that the algorithm models dependencies and relationships between the target output and input features. The AI algorithm, breathing 126 estimates breathing related parameters, such as breath hold duration 270, breath type (deep or shallow) 272, breath rate 274, and breathing impact on target organ 276 from inputs, such as a trigger 260, an ultrasound scan 222, and tracking data 224. The AI algorithm, breathing 126 will be described in greater detail below.

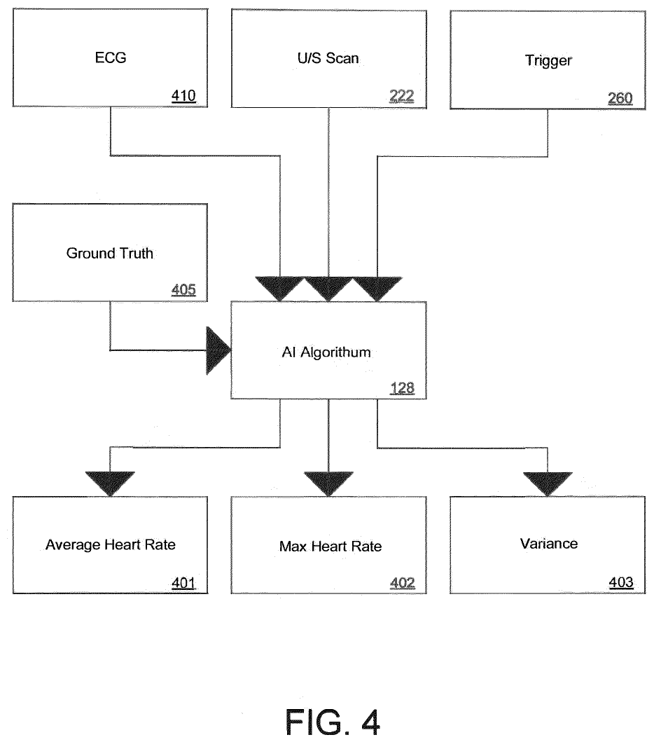

In one embodiment illustrated in FIG. 4, the image analysis module 123 further comprises an AI algorithm, heartbeat 128. The AI algorithm, heartbeat 128 is a regression-based artificial intelligence algorithm, similar to the AI algorithm, breathing 126. The AI algorithm, heartbeat estimates heart rate during an ultrasound scan based on a trigger 260, an ultrasound scan 222, and an electrocardiogram (EKG) 410. The ground truth 405 for heart rate may be provided by user input based on the EKG. The heart rate measurements that are estimated may include: average heart rate 401, maximum heart rate 402, and heart rate variance 403.

A population algorithm 129 is also stored on memory 120. The population algorithm receives the outputs 270, 272, 274, 276, 401, 402, 403 from the AI algorithms 126, 128 and may generate a record such as a survey, questionnaire, patient chart amendment, adjustment to an electronical medical record, or similar down-stream record documentation and alerts. The record can be reviewed and edited by a sonographer, technician, or other medical technician and can be reviewed and uploaded to an electronic medical record system (EMR) 180 either prior to review or in response to review by a reviewer. The EMR may be accessible and operably connected to the processor 110, such as through hardwiring or through the internet.

According to one embodiment, a microphone 160 is operably connected to the processor 110 to provide an audible trigger to initiate image evaluation of breathing patterns. The microphone signal may be input to a word recognition program to detect a signal phrase, such as “take a deep breath and hold it”, for example.

The microphone 160 listens for trigger words/sentences uttered by the imaging technologist/sonographer performing the upstream ultrasound exam (e.g., a request for a breath-hold is usually given verbally, such as “Take a deep breath in and hold”). Later on, words/utterances by the imaging tech that are indicative of the end of the breath-hold request (e.g., “breathe normally now”) can serve as an indicator that the patient should now no longer be holding their breath. These time points can serve as the boundaries within which the frames of the ultrasound image stream can be analyzed to estimate the true breath-hold duration.

Deep learning algorithms can be used to associate a specific audio recording to a sequence of ultrasound images. The audio-recording can be processed in a spectrogram form to capture a two-dimensional representation of sound where time and frequency are included. The deep learning algorithms can also include the periodicity of repeated sentences during an imaging exam. Similarly, other in-between verbal cues can also be processed to provide context to the ultrasound video processing. For example, words or sentences that are indicative of the success or failure of the requested task (e.g., “great job holding your breath, just a few more seconds” or “no problem, let's try this again” etc.). In an example, the present techniques disclose a system, method, or computer-readable medium for extracting breathing pattern data from ultrasound images for aiding downstream clinical patient management. The present techniques may relate to a detection device (160, 123) configured to detect a trigger event indicative of a breathing pattern from at least one of an audio-based trigger and an image-based trigger from an ultrasound video stream. The present techniques may also disclose an image analysis module (123) that that identifies a breathing pattern in the ultrasound video stream responsive to detection of the trigger event, the image analysis module to extract at least one breathing-related parameter. Further, the present techniques may also disclose an algorithm (129) to generate a patient record with the at least one breathing-related parameter. In an example, the detection device is the image analysis module (123) and the trigger event is a specific motion in the ultrasound video stream, wherein the specific motion in the ultrasound video stream comprises at least one of (i) a rate of movement of an identified feature in the video stream and (ii) a landmark moving at least one of entering and exiting an ultrasound image frame. The present techniques may also include the image analysis module receives tracking data (224) for an ultrasound probe (130) synchronized with the video stream (222) and the image analysis module filters out probe motion from the video stream based at least on the tracking data for the ultrasound probe. In this example, the tracking data for the ultrasound probe is generated by an inertial measuring unit (IMU) (132) integral with the ultrasound probe. In an example the present technique may also include uploading the patient record to an electronic medical record (EMR) system (60) with a mitigation step identified and recorded in response to the patient record being uploaded for a procedure impacted by the at least one breathing-related parameter.

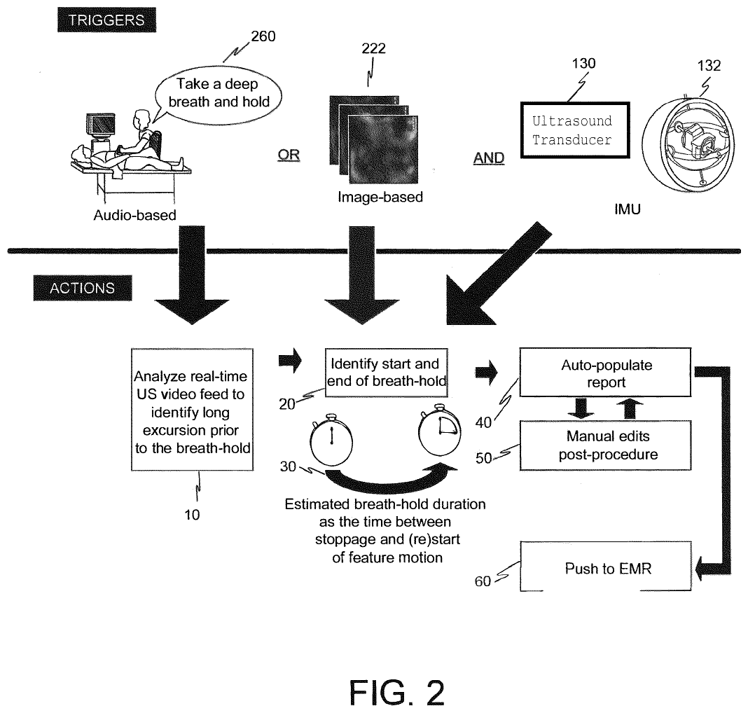

FIG. 2 illustrates a method for extracting breathing pattern data from ultrasound images for aiding downstream clinical patient management according to an embodiment of the presently disclosed techniques. In a first step, a trigger 260 is detected. In one embodiment, the trigger is an audio-based trigger, such as a technician telling a patient to take a deep breath and hold it. As described above, this trigger may be detected by a microphone 160, aided by word recognition software.

Alternatively, an image-based trigger may be used. For example, an ultrasound image stream may be monitored for movement consistent with a large incursion of breath in preparation for a breath-hold. The alternative triggers are shown in FIG. 2 by the disjunctive “or” between the audio-based trigger and the image-based trigger.

In response to detection of the trigger 260, the imaging analysis module 123 analyzes the ultrasound scan 222 (video feed) for motion consistent with a particular breathing pattern. Specifically, according to one embodiment, the imaging analysis module 123 analyzes real-time ultrasound video to identify motion consistent with a long excursion of breath prior to a breath-hold (step 10). FIG. 2 shows the conjunctive “and” between the triggers and the ultrasound probe 130, indicating that an ultrasound video feed is provided in addition to either the audio-based trigger or the image-based trigger. It should be noted that the video stream does not need to be provided in real-time, as an alternate embodiment would be analysis of a stored video stream to estimate breathing related parameters, such as breath hold duration. The imaging analysis module 123 extracts feature motion data 223 using methods of analysis, such as landmark tracking, image correlation, or other methods of analysis. The extraction of feature motion may be aided by an IMU 132 integral with the ultrasound probe 130 as shown in FIG. 2. Then, the imaging analysis module 123 identifies stoppage and restart of feature motion consistent with the start and end of a breath-hold (step 20). Time stamps can be readily obtained for the start and end of the breath-hold. The breath-hold duration is then calculated as the difference between the start and end of the breath-hold (step 30).

The population algorithm 129 automatically populates a record such as a survey, questionnaire, patient chart amendment, adjustment to an electronical medical record, or similar down-stream record documentation and alerts (step 40). The population algorithm may use natural language processing to identify requested data to facilitate auto-population of the record. The record can be reviewed and edited by a sonographer, technician, or other medical technician and can be reviewed and edited (step 50).

The report may be uploaded to the EMR system 180 for reference in future procedures where breath-hold is required to identify a possible need for mitigation steps. The report can also identify the inability to remain still so that stabilizing tools can be made available. In another example the need for special considerations, such as a larger scan room can be identified. Mitigation steps can involve the use of stabilizing or support tools such as leg braces or triangular sponge/foam pads, or the choice of a larger scan room, for example, in the case of the claustrophobic patient.

When there is no motion other than the motion due to breathing in the area of the ultrasound image stream, and landmarks are easily identifiable, motions corresponding to a long excursion of breath prior to breath-hold, stopping motion at the beginning of a breath-hold, and restart of motion at the end of a breath-hold are easily determined. In many situations, however, it can be difficult to analytically differentiate between probe motion and other patient-related motion (such as breathing and/or the inability to remain still), especially when both kinds of motion are intertwined and subtle. That can make it difficult to estimate with certainty whether the patient is or is not successfully holding their breath.

To differentiate probe motion from patient-related motion, the tracking data 224 for the ultrasound transducer 130 may be used to isolate the intentional probe motion, which can then be filtered out of the observed motion, leaving just the patient-related motion.

To further overcome the problem of multiple motion sources, the AI algorithm, breathing 126 is used to distinguish various motions and estimate breathing related parameters. The AI algorithm, breathing 126 is a regression-based AI algorithm that develops a mapping model. That is, it models the mapping of the data inputs: trigger 260 from microphone 160 (or image based trigger), ultrasound image stream 222 from ultrasound system 100, and tracking data 224 from IMU 132 (or electromagnetic tracking data), which collectively define a patient state to one or more estimated breathing-related parameters, such as breath-hold duration 270. The estimated breathing parameters may also include breath type 272, breathing rate 274, and breathing impact on target organ 276.

For estimating breath-hold duration, the AI model can be trained as a regression model, for the following situations:
No probe or patient (breathing or otherwise) motion: The ultrasound image content, in this case, will more or less stay constant, except for cardiac motion (which may or may not be visible in the images, depending on the anatomy being scanned).
Patient holds breath but cannot stay still; no intentional probe motion.
Patient releases breath-hold early, no intentional probe motion.
Patient cannot stay still and also releases breath-hold early, no intentional probe motion.
Breathing motion and intentional probe motion.
Other combinations.

During a training phase, the AI algorithm 126 develops a model for predicting breathing-related parameter(s) using a machine learning approach. The algorithm estimates the parameter(s) and compares the estimated parameter(s) to a ground truth. Following the training phase, the model is used to estimate the parameter(s) without a ground truth.

A ground truth 290 is provided to the AI algorithm, breathing 126. The ground truth comprises actual measurements of the breathing parameters. The ground truth 290 used during the training phase of the AI algorithm, breathing 126 can come from operator input entered through a user interface 150 and/or from add-ons such as respiration belts, cameras etc. The operator input can include identification of the specific ultrasound image frames that corresponded to events like the loss of breath-hold, sudden patient movement, start and stop of probe motion etc. Note that such equipment will only be needed for the training phase and will not be needed when the trained model is in operation. Also, in order to differentiate the different breathing patterns, the AI algorithm, breathing 126 can be trained in learning differences between deep or shallow breathing, as well as predicting the breath-hold duration.

During the training phase, the AI algorithm, breathing 126 sequentially predicts the breath-hold duration (and other breathing parameters) based on the inputs 260, 222, 224 and the mapping model and updates the mapping model based on the ground truth 290. The AI algorithm, breathing 126 applies an optimization policy of minimizing the difference between the estimated parameters and the ground truth to adjust the mapping model, thereby improving its predictions over time.

After the training phase, the AI algorithm, breathing 126 continues to provide a prediction breath-hold duration estimate for each new patient or exam. The AI algorithm, breathing may also be trained to provide other breathing related parameters, such as:
Breathing type (example labels could be shallow/medium/deep),
Breathing rate (example labels could be slow/medium/fast), and
Breathing impact on target organ in terms of organ motion (example labels could be high/medium/low) for example breathing movement may impact the assessment of ribs or thorax generally more than breathing could impact movement of feet.

The AI algorithm, heartbeat 128 may be trained to estimate the heart rate related parameters, such as average heart rate, maximum heart rate, and heart rate variance during an ultrasound scan. The AI Algorithm, heartbeat 128 uses a regression algorithm similar to the AI Algorithm, breathing. The ground truth for the AI Algorithm heartbeat may be provided by an EC input to the ultrasound system.

The populate algorithm 129 auto-populates a report with the extracted parameters (breath-hold duration (max and average, throughout that imaging scan), breathing type, breathing rate, or other parameters) can be used to populate a report. Such a report is also editable by the imaging technologist, who can override any of the automatically determined values.

The report can be presented to the patient before new, follow-up, or downstream imaging exams and can also be examined by the technologist before starting and planning the downstream imaging sequence or scanning protocol. The report can become part of the patient EMR in the future.

Upon confirmation by the imaging technologist, the report can be pushed to a central database/server (60) that is accessible by software on systems that carry out subsequent downstream imaging (US/MR/other) or other procedures on that patient.

Any ‘abnormal’ findings in the report (e.g. inability to hold breath for >3 secs, inability to remain still) can be highlighted. Highlighted findings can be displayed to the operator of downstream exams for the same patient, or can be displayed in the institution's scheduling system when a downstream exam for this patient is being scheduled.

The presently disclosed techniques can take the form of an entirely hardware embodiment, an entirely software embodiment or an embodiment containing both hardware and software elements. In an exemplary embodiment, the presently disclosed techniques is implemented in software, which includes but is not limited to firmware, resident software, microcode, etc.

Furthermore, the presently disclosed techniques may take the form of a computer program product accessible from a computer-usable or computer-readable medium providing program code for use by or in connection with a computer or any instruction execution system or device. For the purposes of this description, a computer-usable or computer readable medium may be any apparatus that can contain, store, communicate, or transport the program for use by or in connection with the instruction execution system, apparatus, or device.

The foregoing method may be realized by a program product comprising a machine-readable media having a machine-executable program of instructions, which when executed by a machine, such as a computer, performs the steps of the method. This program product may be stored on any of a variety of machine-readable media, including but not limited to compact discs, floppy discs, USB memory devices, and the like. Moreover, the program product may be in the form of a machine-readable transmission such as blue ray, HTML, XML, or the like.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk an optical disk. Current examples of optical disks include compact disk-read only memory (CD-ROM), compact disk-read/write (CD-R/W) and DVD.

The preceding description and accompanying drawing are intended to be illustrative and not limiting of the presently disclosed techniques. The scope of the presently disclosed techniques are intended to encompass equivalent variations and configurations to the full extent of the following claims.

### REFERENCE NUMBER LISTING

10 analyze ultrasound video
20 identify start and end of breath-hold
30 estimate breath-hold duration
40 auto-populate report
50 manual edits
60 push to EMR
100 ultrasound system
110 processor
120 memory
122 image processing module
123 image analysis module
124 tracking module
126 artificial intelligence (AI) algorithm, breathing
128 artificial intelligence (AI) algorithm, heartbeat
129 population algorithm
130 ultrasound transducer
132 inertial measurement unit (IMU)
140 display
150 user interface, input
160 microphone
222 ultrasound scan (image)
223 feature motion data
224 tracking data
260 trigger, audio
270 breath hold duration
272 breathing type
274 breathing rate
276 breathing impact on target organ
280 heart rate
290 ground truth data
401 average heart rate
402 maximum heart rate
403 heart rate variance
410 EKG
405 ground truth, heartbeat
510 subtract tracking data from feature motion
520 patient-related motion

1. A system for extracting breathing pattern data from ultrasound images for aiding downstream clinical patient management, comprising:
a detection device configured to detect a trigger event indicative of a breathing pattern from at least one of an audio-based trigger and an image-based trigger from an ultrasound video stream;
an image analysis module that that identifies a breathing pattern in the ultrasound video stream responsive to detection of the trigger event, the image analysis module to extract at least one breathing-related parameter; and
an algorithm to generate a record with the at least one breathing-related parameter.

2. The system of claim 1, wherein the image analysis module comprises an artificial intelligence (AI) algorithm trained to estimate the at least one breathing-related parameter.

3. The system of claim 2, wherein the artificial intelligence algorithm is a model-based regression algorithm.

4. The system of claim 1, wherein the detection device is a microphone and the triggering event is a spoken word.

5. The system of claim 1, wherein the detection device is the image analysis module and the trigger event is a specific motion in the ultrasound video stream, wherein the specific motion in the ultrasound video stream comprises at least one of (i) a rate of movement of an identified feature in the video stream and (ii) a landmark location being detected as moving such that it is at least one of entering and exiting an ultrasound image frame.

6. The system of claim 1, wherein the image analysis module receives tracking data for an ultrasound probe synchronized with the video stream and the image analysis module filters out probe motion from the video stream based at least on the tracking data for the ultrasound probe.

7. The system of claim 6, wherein the tracking data for the ultrasound probe is generated by an inertial measuring unit (IMU) integral with the ultrasound probe.

8. The system of claim 1, wherein the estimated breathing parameters comprise breath hold duration.

9. The system of claim 1, wherein the estimated breathing parameters comprise breathing type.

10. The system of claim 1, wherein the estimated breathing parameters comprise breathing rate.

11. The system of claim 1, wherein the estimated breathing parameters comprise breathing impact on a target organ.

12. The system of claim 1, further comprising an artificial intelligence algorithm trained to estimate heartbeat-related parameters.

13. A method for extracting breathing pattern data from ultrasound images for aiding downstream clinical patient management, comprising the steps of:
detecting a trigger event indicative of a breathing pattern from at least one of an audio-based trigger and an image-based trigger from an ultrasound video stream;
identifying a breathing pattern in the ultrasound video stream responsive to detection of the trigger event;
extracting at least one breathing-related parameter; and
generating a record with the at least one breathing-related parameter.

14. The method of claim 13, further comprising the step of:
uploading the record to an electronic medical record (EMR) system with a mitigation step identified and recorded in response to the record being uploaded for a procedure impacted by the at least one breathing-related parameter.

15. A computer program product comprising a machine-readable media having encoded thereon program code executable by a processor to perform the steps of:
detecting a trigger event indicative of a breathing pattern from at least one of an audio-based trigger and an image-based trigger from an ultrasound video stream;
identifying a breathing pattern in the ultrasound video stream responsive to detection of the trigger event;
extracting at least one breathing-related parameter; and
generating a record with the at least one breathing-related parameter.
